(19) United States
(12) Patent Application Publication
(10) Pub. No.: US 2020/0376097 A1
(43) Pub. Date: Dec. 3, 2020

(54) PHARMACEUTICAL COMPOSITION, USE AND METHOD FOR FISTULA TREATMENT IN CHILDREN

(71) Applicant: Shin-Kong Wu Ho-Su Memorial Hospital, Taipei City (TW)

(72) Inventor: Chi-Jen CHANG, NEW TAIPEI CITY (TW)

(21) Appl. No.: 16/887,636

(22) Filed: May 29, 2020

(30) Foreign Application Priority Data
May 29, 2019 (TW) 108118623

(51) Int. Cl. A61K 38/48; A61K 38/36; A61K 38/45; A61K 9/08; A61K 47/02

(52) U.S. Cl. CPC A61K 38/4833; A61K 38/363; A61K 47/02; A61K 9/08; A61K 38/45

(57) ABSTRACT

The disclosure provides a pharmaceutical composition for fistula treatment in children. The pharmaceutical composition includes a fibrinogen, a thrombin and a coagulation factor XIII. Wherein a dose of the fibrinogen is between 20 mg/mL and 200 mg/mL. A dose of the thrombin is between 1 IU/mL and 20 IU/mL. A dose of the coagulation factor XIII is less than or equal to 10 IU/mL. The fistula in children is anal fistula or urethra fistula. The disclosure further provides a use and a method of the pharmaceutical composition for fistula treatment in children. The application of the pharmaceutical composition, the use and the method of the present invention are advantageous for fistula treatment in children.

### CROSS REFERENCE TO RELATED APPLICATIONS

This Non-provisional application claims priority under 35 U.S.C. § 119(a) on Patent Application No(s). 108118623 filed in Taiwan, Republic of China on May 29, 2019, the entire contents of which are hereby incorporated by reference.

### BACKGROUND OF THE INVENTION

### Field of Invention

The disclosure relates to a pharmaceutical composition, a use and a method for fistula treatment in children.

### Related Art

Fistula refers to an abnormal tract or connection between body organs or blood vessels. Take anal fistula as an example, anal fistula forms between the rectum and a part of the intestine (or other organs), or forms between the rectum and perianal skin. Except to cause severe pain, anal fistula often causes infection and suppuration. In addition, anal fistula also may cause feces to leak from the rectum.

Anal fistula often occurs in males, with a male to female ratio between 2:1 and 7:1. The age of patients with anal fistula ranges from newborn babies to the elderly, and is most common in middle-aged people between 30 and 50 years old. Anal fistula may be formed due to disease or infection in adults. For example, the anal fistula may be caused by anal gland obstruction which leads to the formation of an abscess, and then the abscess further cause perforation from the rectum to perianal skin and subsequently formed the anal fistula. The fistula path of adult follows Goodsall's rule, which means that the anterior portion of anal fistula is a linear path from the external opening to the internal opening, the posterior portion of anal fistula is a curve path from the external opening to the internal opening, and the internal opening often located at the posterior wall of the anal canal.

Unlike in adults, anal fistula in children majorly caused by the congenital abnormality of the anal gland. The fistula path of children does not follow Goodsall's rule. The fistula in children extends radially from the external opening to the anal canal (involved crypt recess), thus the fistula path of children is a linear path. Common causes of anal fistula in children include the androgen excess in the mother's uterus during the fetal period resulting in abnormal gland development, epithelial cell ectopic, irregular hypertrophy of the dental line, and deep crypt that is easy to hide bacteria. Most fistula patients are male infants under 12 months of age, and the fistula often not be too long.

The anal fistula can be treated by surgical treatment (for example, fistulectomy). However, there are many side effects would occur after the patients who were treated by surgery, such as prolonged healing, anal fistula recurrence and anal incontinence. In addition, if the patient is a child with a smaller fistula than that in adults, the surgical treatment would be more difficult.

Accordingly, it is an urgent need to provide a pharmaceutical composition for fistula treatment in children, the use and the method thereof. The pharmaceutical composition can enhance the success rate, and decrease the difficulty of fistula treatment, and thus avoid the side effects including prolonged healing, anal fistula recurrence, and anal incontinence.

### SUMMARY OF THE INVENTION

In view of the foregoing objectives, the purpose of the disclosure is to provide a pharmaceutical composition for fistula treatment in children, the use and the method thereof. The pharmaceutical composition can enhance the success rate, and decrease the difficulty of fistula treatment, and thus avoid the side effects including prolonged healing, anal fistula recurrence, and anal incontinence.

To achieve the above objective, the disclosure provides a pharmaceutical composition for use in fistula treatment in children, wherein the pharmaceutical composition comprises a fibrinogen, a thrombin and a coagulation factor XIII. The dose of the fibrinogen is between 20 mg/mL and 200 mg/mL. The dose of the thrombin is between 1 IU/mL and 20 IU/mL. The dose of the coagulation factor XIII is less than or equal to 10 IU/mL. The fistula in children is anal fistula or urethra fistula.

To achieve the above objective, the disclosure also provides a pharmaceutical composition for fistula treatment in children, wherein the pharmaceutical composition comprises a fibrinogen, a thrombin and a coagulation factor XIII. The dose of the fibrinogen is between 20 mg/mL and 200 mg/mL. The dose of the thrombin is between 1 IU/mL and 20 IU/mL. The dose of the coagulation factor XIII is less than or equal to 10 IU/mL. The fistula in children is anal fistula or urethra fistula.

To achieve the above objective, the disclosure further provides a method for fistula treatment in children. The method comprises a step of applying a pharmaceutical composition to the fistula in children, wherein the pharmaceutical composition comprises a fibrinogen, a thrombin and a coagulation factor XIII. The dose of the fibrinogen is between 20 mg/mL and 200 mg/mL. The dose of the thrombin is between 1 IU/mL and 20 IU/mL. The dose of the coagulation factor XIII is less than or equal to 10 IU/mL. The fistula in children is anal fistula or urethra fistula.

In one embodiment, the pharmaceutical composition is applied to the fistula in children by filling the inside of fistula from one end until another end is filled.

In one embodiment, the pharmaceutical composition is applied to the fistula in children by using a syringe.

In one embodiment, the fibrinogen and the thrombin are mixed to form the pharmaceutical composition just before the pharmaceutical composition is applied to the fistula in children.

In one embodiment, the pharmaceutical composition is applied to the fistula in children as a liquid dosage form or a gel dosage form.

In one embodiment, a volume mixing ratio of the fibrinogen and the thrombin is 1:1.

In one embodiment, the pharmaceutical composition further comprises a pharmaceutically acceptable carrier, the pharmaceutically acceptable carrier is water, phosphate buffer, glycerol, sucrose ester, gelatin or polysorbate.

In one embodiment, the pharmaceutical composition further comprises a calcium chloride solution.

In one embodiment, the fibrinogen is a human fibrinogen purified from a nature source, a transgene-human fibrinogen or a recombinant human fibrinogen.

In one embodiment, the thrombin is a human thrombin purified from a nature source, a transgene-human thrombin or a recombinant human thrombin.

In one embodiment, the pharmaceutical composition is used for children under or equal to 12 years old.

As mentioned above, the efficacy of this disclosure is to provide a pharmaceutical composition for fistula treatment in children, the use and the method thereof. The pharmaceutical composition can enhance the success rate, and decrease the difficulty in fistula treatment, and thus avoid the side effects including prolonged healing, anal fistula recurrence, and anal incontinence.

### BRIEF DESCRIPTION OF THE DRAWINGS

FIG. 1A is a schematic view of the fistula in children.

FIG. 1B is a schematic view of the fistula in adults.

FIG. 2 is a schematic view of the combination of the pharmaceutical composition of this invention and a syringe.

FIG. 3A is a schematic view of the pharmaceutical composition of this invention used for fistula treatment.

FIG. 3B is another schematic view of the pharmaceutical composition of this invention used for fistula treatment.

### DETAILED DESCRIPTION OF THE INVENTION

The embodiments and examples of the pharmaceutical composition for fistula treatment, the use and the method thereof in this invention will be apparent from the following detailed description, which proceeds with reference to the accompanying figures, wherein the same references relate to the same elements.

The pharmaceutical composition, the use and the method thereof can enhance the success rate and decrease the difficulty in fistula treatment in children, and thus avoid the side effects including prolonged healing, anal fistula recurrence, and anal incontinence.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only, and is not intended to be limiting.

The term “fistula” refers to a pathological tract which formed between the epithelial cells of the body surface and internal organs or deep tissues. The fistula has an external opening and an internal opening. Depending on the location of the formation of the fistula, it can be classified as anal fistula or urethra fistula. For example, anal fistula forms between the rectum and a part of the intestine (or other organs), or forms between the rectum and perianal skin.

The term “child” refers to a human before adolescence which can be classified as the following periods according to the age of the child: fetal, neonate, infant, early child, preschool child, and school-age child. The term “fetal” refers to a human between the period of conception and parturition. The term “neonate” refers to a human within 28 days after birth. The term “infant” refers to a human being between 28 days after birth and 1 year old. The term “early child” refers to a human being between 1 and 3 years old. The term “preschool child” refers to a human being between 3 and 7 years old. The term “school-age child” refers to a human being between 7 and 12 years old. As used herein, the term “child” includes but not limited to neonate, infant, early child, preschool child, and school-age child.

The term “fibrinogen” refers to a water-soluble protein. When platelets rupture, fibrinogen can be converted into water-insoluble fibrin by thrombin. Fibrin can combine with other blood cells to form a mass and coagulate into a blood clot, so as to strengthen the thrombus formed by platelets. As used herein, “fibrinogen” includes but not limited to human fibrinogen purified from a nature source, transgene-human fibrinogen, recombinant human fibrinogen, or the combination thereof.

The term “thrombin” refers to a protein which can convert soluble fibrinogen in plasma into insoluble network-like fibrin, and thus causes blood to clot. Thrombin is generated by the hydrolysis of prothrombin. As used herein, “thrombin” includes but not limited to human thrombin purified from a nature source, transgene-human thrombin, recombinant human thrombin, or the combination thereof.

As used herein, the term “pharmaceutically acceptable carrier” means a pharmaceutically acceptable salts, material, composition or carrier, such as a filler, diluent, excipient, or encapsulating material, involved in applying the pharmaceutical composition useful within the invention to the organ or tissue of the subject such that it may perform its intended function. Each salt or carrier must be compatible with the other ingredients of the formulation, including the fibrinogen and the thrombin useful within the invention, and not injurious to the subject. Some examples of materials that may serve as pharmaceutically acceptable carriers include: cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; sugars, such as lactose, glucose and sucrose; glycols, such as propylene glycol; polyols, such as glycerin, sorbitol, mannitol and polyethylene glycol; starches, such as corn starch and potato starch; esters, such as ethyl oleate and ethyl laurate; agar; buffering agents, such as magnesium hydroxide and aluminum hydroxide; alginic acid; granulating agents; wetting agents; lubricating agent; surface active agents; binding agents; diluent; disintegrating agents; emulsifying agents; coloring substances; releasing agents; aromatic agents; preservatives; gelling agents; antioxidants; coating agents; plasticizers; thickening agents; stabilizers; hardening agents; setting agents; humectant; carriers; and other non-toxic compatible substances employed in pharmaceutical formulations.

The terms “factor” and “coagulation factor” refer to the substances involved in coagulation which are named as roman numerals by orders. The activated factor is marked by “a” at the lower right position of its name. The physiological role of factor is to be activated when bleeding, it can adhere to platelets and to plug the leaks of blood vessels. “Factors” include but not limited to factor I, factor II, factor III, factor IV, factor V, factor VII, factor IX, factor X, factor XI, factor XII and factor XIII, wherein factor I is fibrinogen, and factor II is thrombin.

As used herein, the terms “patient” and “subject” can be used interchangeably and may refer to a human or non-human mammal. Non-human mammals include, for example, livestock and pets, such as ovine, bovine, porcine, canine, feline and murine mammals. Preferably, the patient is human.

The term “coagulation” refers to the status that the fibrinogen is converted into water-insoluble fibrin by the thrombin, and then the fibrin gathers with each other and becomes unable to flow.

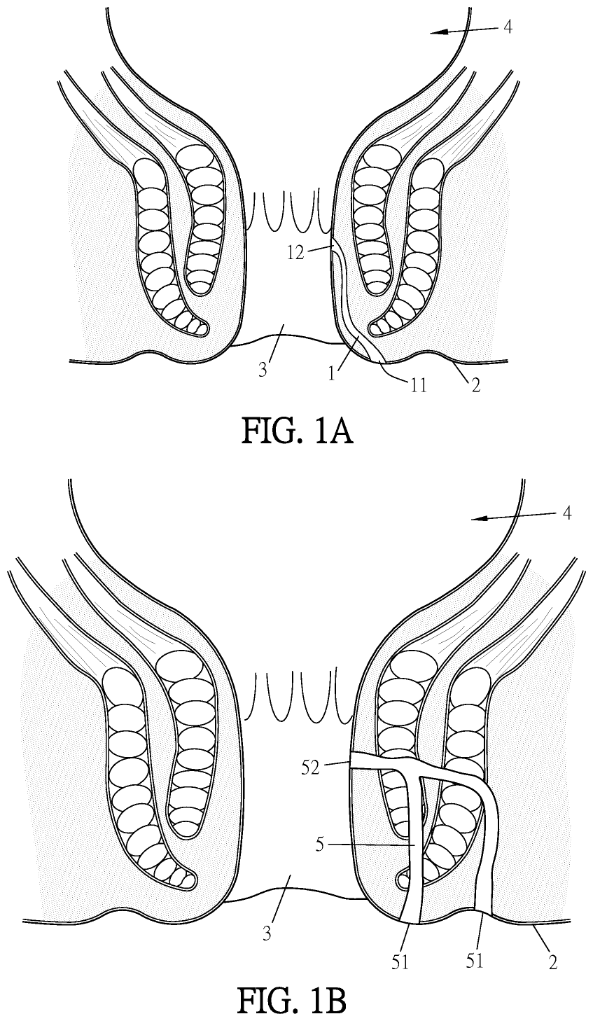

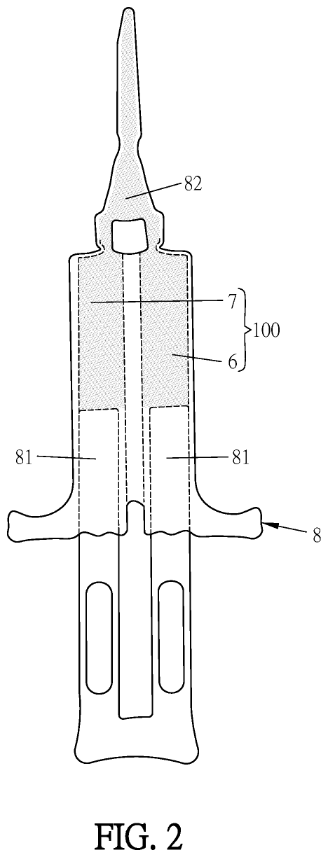

Please refer to FIG. 1A, FIG. 1A is a schematic view of the fistula in children to illustrate the morphology of the fistula in children. Although here take the anal fistula in children for example, the fistula in children also can be the urethra fistula, or other fistulae in children known by a person in the art, and FIG. 1A is not intended to be limiting. As shown in FIG. 1A, the fistula (the anal fistula) in children 1 refers to a pathological tract which formed between the body surface 2 and the anal 3. The fistula in children 1 has an external opening 11 and an internal opening 12, and the anal 3 is connected to the rectum 4.

Please refer to FIG. 1B, FIG. 1B is a schematic view of the fistula in adults. The differences between the fistula in adults 5 and the fistula in children 1 include: 1) the structures of the fistula in adults are complex than that in children, and the tracts of fistula can be longer and the path can be curved; 2) the fistula in adults may have many openings (for example, two external openings 51 and one internal opening 52). However, the fistula in children 1, as shown in FIG. 1A, has a simpler structure, the tract of fistula is shorter and the path is linear. The pharmaceutical composition of this invention, the use and the method thereof are used for treating the fistula in children 1 instead of the fistula in adults 5, and the reasons are described as following.

Please refer to FIG. 2, FIG. 2 is a schematic view of the combination of the pharmaceutical composition of this invention and a syringe. In this embodiment, the pharmaceutical composition 100 comprises a fibrinogen 6 and a thrombin 7. The syringe 8 has two chambers 81, and the fibrinogen 6 and the thrombin 7 are separated in different chambers 81. In particular, although here is take the syringe for containing the fibrinogen 6 and the thrombin 7 for example, there are other containers can be used for containing the pharmaceutical composition, such as, but not limited to test tubes, beakers, flasks, ampoules, serum bottles, or other containers can be used for containing the pharmaceutical composition known by a person in the art, and FIG. 2 is not intended to be limiting.

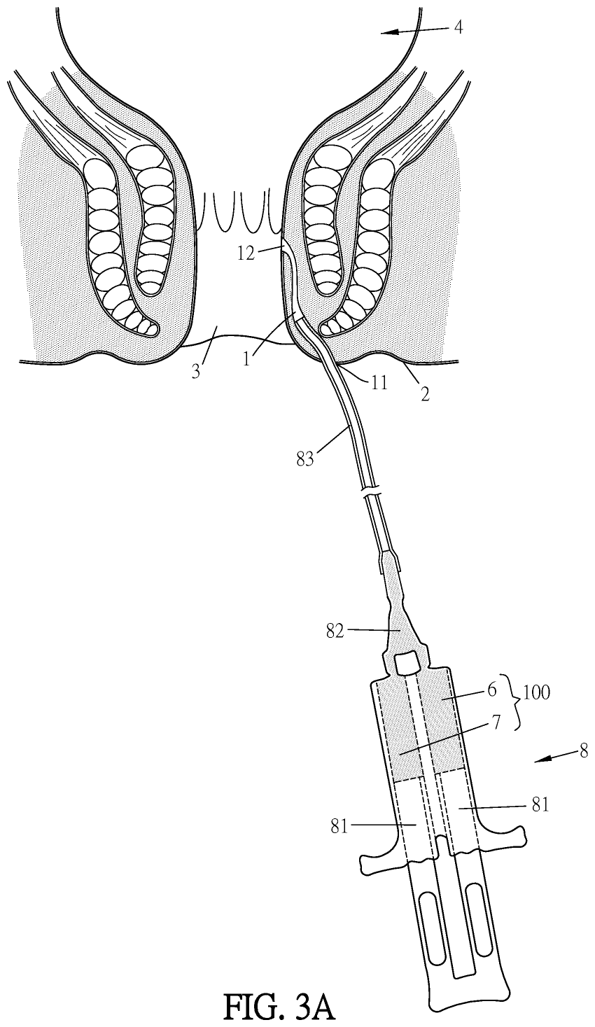

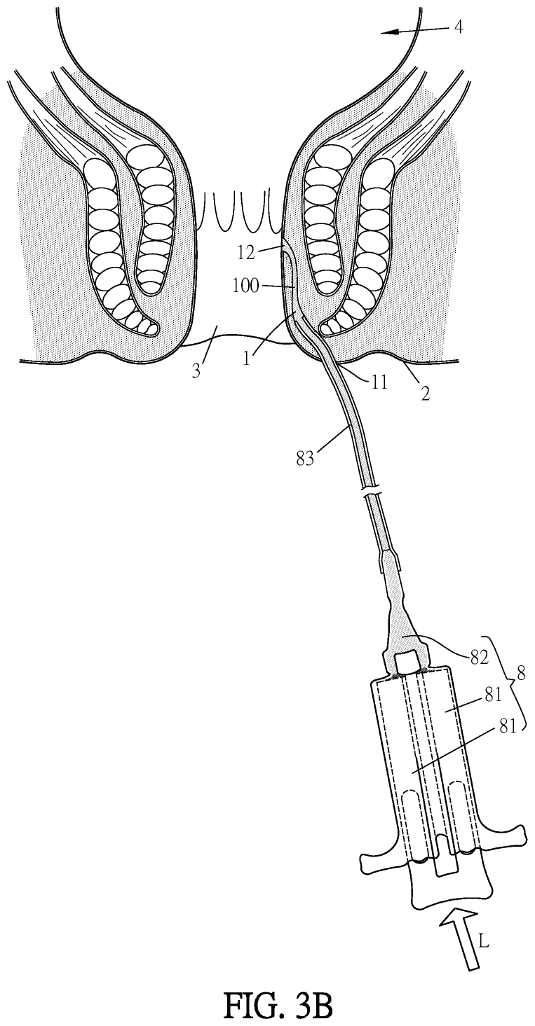

Please refer to FIGS. 3A and 3B for illustrating how the pharmaceutical composition 100 is used for treating the fistula in children 1. FIG. 3A is a schematic view of the pharmaceutical composition of this invention used for fistula treatment. FIG. 3B is another schematic view of the pharmaceutical composition of this invention used for fistula treatment. As shown in FIG. 3A, the pharmaceutical composition 100 is applied to the fistula (the anal fistula) in children 1 by using a syringe 8. In more detailed, please refer to FIGS. 2 and 3A, the fibrinogen 6 and the thrombin 7 are separated in different chambers 81 of the syringe 8, the fibrinogen 6 and the thrombin 7 are mixed at the front end 82 of the syringe 8 with a ratio of 1:1 by volume to form the pharmaceutical composition 100 just before the pharmaceutical composition 100 is applied to the fistula (the anal fistula) in children 1. Herein, the pharmaceutical composition 100 is liquid dosage form or gel dosage form. Then, as shown in FIG. 3B, a catheter 83 is connected to the front end 82 of the syringe 8, and the catheter 83 is inserted into the external opening 11 of the fistula (the anal fistula) in children 1. After that, the syringe 8 is pushed along with the direction of arrow L, the pharmaceutical composition 100 is applied to the fistula (the anal fistula) in children 1 by filling the inside of fistula from one end (the external opening 11) until another end (the internal opening 12) is filled. Thus, the pharmaceutical composition 100 is filled in the entire fistula (the anal fistula) in children 1. In addition, when the fibrinogen 6 and the thrombin 7 are contained in other containers instead of the syringe, the fibrinogen 6 and the thrombin 7 can be mixed in those containers first, and then the mixture is transferred in the syringe, so as to use the syringe for applying the mixture to the fistula (the anal fistula) in children 1. It is to be understood that after the fibrinogen 6 and the thrombin 7 are mixed to be the pharmaceutical composition 100, the pharmaceutical composition 100 should be applied to the fistula (the anal fistula) in children 1 before it is coagulated. In particular, although the figures show that the catheter 83 is connected to the front end 82 of the syringe 8 and then the catheter 83 is inserted into the external opening 11 of the fistula (the anal fistula) in children 1, the front end 82 of the syringe 8 can also be inserted into the external opening 11 of the fistula (the anal fistula) in children 1 directly without the catheter 83 for applying the pharmaceutical composition, and FIGS. 3A and 3B are not intended to be limiting.

In this embodiment, the dose of the fibrinogen 6 is between 20 mg/mL and 200 mg/mL, and the dose of the thrombin 7 is between 1 IU/mL and 20 IU/mL. In a preferred embodiment, the dose of the fibrinogen 6 is between 10 mg/mL and 100 mg/mL, and the dose of the thrombin 7 is between 2 IU/mL and 10 IU/mL. In another preferred embodiment, the dose of the fibrinogen 6 is between 30 mg/mL and 60 mg/mL, and the dose of the thrombin 7 is between 2 IU/mL and 6 IU/mL. In a most preferred embodiment, the dose of the fibrinogen 6 is between 35 mg/mL and 55 mg/mL, and the dose of the thrombin 7 is between 2 IU/mL and 3 IU/mL. In more detailed, in this embodiment, for the purpose that filling the entire fistula (the anal fistula) in children 1, the doses of the fibrinogen 6 and the thrombin 7 make the mixture coagulated after a period. Therefore, the mixture can be fully filling the fistula (the anal fistula) in children 1 before the fibrinogen 6 and the thrombin 7 are coagulated. Preferably, the period is longer than or equal to 30 seconds. Preferably, the period is longer than or equal to 1 minute. Preferably, the period is longer than or equal to 3 minutes.

In this embodiment, the pharmaceutical composition 100 further comprises a pharmaceutically acceptable carrier. The pharmaceutically acceptable carrier can carry or transfer the fibrinogen 6 and the thrombin 7 to the fistula of the patient such that the fibrinogen 6 and the thrombin 7 may perform their intended functions in treating the fistula. The pharmaceutically acceptable carrier is, such as, but not limited to water, phosphate buffer, glycerol, sucrose ester, gelatin, or polysorbate.

In this embodiment, the pharmaceutical composition 100 further comprises a calcium chloride solution and a coagulation factor. For example, the coagulation factor is, such as, but not limited to factor I, factor II, factor III, factor IV, factor V, factor VII, factor IX, factor X, factor XI, factor XII and factor XIII. The calcium chloride solution and the coagulation factors can mediate the coagulation of the fibrinogen 6 and the thrombin 7.

In this embodiment, the fibrinogen is human fibrinogen purified from a nature source, transgene-human fibrinogen or recombinant human fibrinogen. The thrombin is human thrombin purified from a nature source, transgene-human thrombin or recombinant human thrombin.

In this embodiment, the fistula in children 1 is anal fistula or urethra fistula. The pharmaceutical composition is used for children under or equal to 12 years old.

In another embodiment, this invention also provides a pharmaceutical composition 100 for use in fistula treatment in children. In addition, this invention further provides a method for fistula treatment in children. The method includes a step of applying a pharmaceutical composition 100 to the fistula in children 1. The concentration or dose of the pharmaceutical composition 100, the types of the carriers, and other properties are mostly the same as those of the pharmaceutical composition 100 described above, and therefore is omitted here for conciseness.

The above embodiments are illustrations that the anal fistula treatment in children as example. However, the pharmaceutical composition, the use and the method thereof can also be used for treating other fistula in children, such as, but not limited to, urethra fistula, and is not intended to be limiting.

To illustrate the efficacy of the use, the method and the pharmaceutical composition of this invention for fistula treatment in children there are several examples shown below.

Material and Method

Patients in this Study

Children with recurrent perianal abscesses (three or more times) who had treated at Shin Kong Wu Ho-Su Memorial Hospital in Taiwan from September 2009 to September 2015 with antibiotics. Patients were referred from the outpatient pediatric department of pediatrics institution to the surgery department for possible surgical management. Patients with a prior incision and drainage of an abscess were excluded from this study. Patients with a history of inflammatory bowel disease (IBD) were also excluded. The informed consent to participate which are approved by the Institutional Review Board (IRB) of the hospital are signed by the parents or guardians of the children.

Fistulectomy

Fistulectomy was performed with patients in the lithotomy position and under general anesthesia. A lubricated retractor was inserted in the anus to determine the origin of the fistula. The abscess is incised and drained. A silver probe was then gently inserted into the opening of the tract to visualize the opening in the anal canal. A scalpel or electrocautery was used to open the fistula tract (please refer to the method described in Spitz L et al. Operative Pediatric Surgery, 6th Ed. Boca Raton, Fla.: CRC Press, 2013, pp 602.). Granulation tissue in the floor of the fistula or in the abscess cavity was curetted as necessary. The overlapping skin was excised to assure free drainage of the tract.

The Preparation of the Pharmaceutical Composition

The pharmaceutical composition of this invention was prepared by using fibrin glue (TISSEEL®, Baxter Healthcare Corporation, Westlake Village, Calif.). There were two major components in fibrin glue, one was TISSEEL solution (contained 72-110 mg/mL of fibrinogen, ≤10 IU/mL of coagulation factor XIII, 3000 KIU/mL of aprotinin and 0.6-1.9 mg/mL of polysorbate 80), and the other one was thrombin solution (contained 500 IU/mL of thrombin and 40 μM/mL of calcium chloride). First of all, 1 mL of the thrombin solution was diluted with 100 mL of the phosphate buffer. Then, 2 mL of 10% (W/V) calcium chloride solution was added to the previous solution and well mixed. 1 mL of the mixture was transferred to another container, and 1 mL of TISSEEL solution was added. 1 minute after added the TISSEEL solution, the mixture was coagulated. Herein, the final concentration of the components of the pharmaceutical composition were: 36-55 mg/mL of fibrinogen, ≤5 IU/mL of coagulation factor XIII, 1500 KIU/mL of aprotinin, 0.3-0.95 mg/mL of polysorbate 80, 2.43 IU/mL of thrombin and 20 μM/mL of calcium chloride. In particular, the thrombin solution also could be diluted with 75 mL, 50 mL or 25 mL of the phosphate buffer depended on the status of the fistula (site, such as high or low; or severity) to adjust the coagulation time of the pharmaceutical composition.

The Method of this Invention for Fistula Treatment Using the Pharmaceutical Composition

The pharmaceutical composition treatment was performed with patients under general anesthesia. An examination was performed to identify the external and internal openings of the fistula. A flexible narrow catheter of 3.5 mm diameter was inserted through the external opening through the fistula toward the internal opening. The thrombin solution was prepared as the above-mentioned method, and then 1 mL of the thrombin solution was added into one of the chamber of a double-chamber syringe. 1 mL of TISSEEL solution was added into the other one chamber of a double-chamber syringe (please refer to the chamber 81 of the syringe 8 in FIG. 2). The front end of these two chambers were both connected to an adjoining piece (please refer to the front end 82 of the syringe 8 in FIG. 2). The double-chamber syringe was used to deliver the TISSEEL solution and the thrombin solution, using the adjoining piece to connect the syringe with the 40-mm-long application cannula. The single-outlet design allows equal volumes of the two solutions to pass through the adjoining piece into the application cannula for thorough mixing. The pharmaceutical composition (2 mL) was injected with the simultaneous slow withdrawal of the catheter until the fistula was completely filled and both openings were sealed.

Follow-Up

All patients were seen in the outpatient department one day after surgery, with successive follow-up at one week and one month. Patients were then examined at three-month intervals until reaching at least two years follow-up observation.

The Comparison of the Treatment Result of Fistula Treatment in Children by Fistulectomy and that by the Pharmaceutical Composition

The patients with fistula in children were treated by the method of prior art or the pharmaceutical composition. In this example, the method of prior art was fistulectomy. The pharmaceutical composition is applied to the fistula in children by using a syringe. A total of 34 children were included in the study; 27 received fistulectomy and 7 received the pharmaceutical composition treatment of this invention. The patient demographic characteristics and treatment results are listed in the following Table 1.

TABLE 1
patient demographic characteristics and treatment results

| | | | Pharmaceutical | | |
|---|---|---|---|---|---|
| | Fistulectomy | | composition treatment | | P value |
| Patient number | 27 | | 7 | | |
| Gender | | | | | 0.374 |
| Male | 26 | (96.3%) | 6 | (85.71%) | |
| Female | 1 | (3.7%) | 1 | (14.29%) | |
| Age (months) | 8 | (1-180) | 14 | (4-144) | 0.522 |
| Follow-up | 1 | (1-7) | 5 | (1-26) | 0.003 |
| (months) | | | | | |
| Result | Healed | | Healed | | |
| Recurrence rate | 1 | (3.7%) | 0 | (0%) | 1.000 |

| | | |
|---|---|---|
| Complications | None | None |

As shown in Table 1, no significant differences in demographic or clinical characteristics (age, gender) were found between the fistulectomy group and the pharmaceutical composition treatment group (P>0.05). And the results of these two groups were both healed. Median follow-up duration was significantly higher in the pharmaceutical composition treatment group compared with that in the fistulectomy group (5 months vs 1 month, P=0.003). In addition, there was one recurrence in the fistulectomy group, the recurrence rate was 3.7% in the fistulectomy group and no recurrences in the pharmaceutical composition treatment group. In other words, the success rate was 96% in the fistulectomy group and the success rate was 100% in the pharmaceutical composition treatment group. As shown in the above-mentioned results, the pharmaceutical composition treatment of this invention can enhance the success rate of fistula treatment in children, which can avoid recurrence, and thus can keep the function of sphincter and reduce the complication of surgery.

The characteristics of seven children who received the pharmaceutical composition treatment are summarized in the following Table 2.

TABLE 2
characteristics of seven children with anal fistula
treated with the pharmaceutical composition

| | | | | |
|---|---|---|---|---|
| | | Fistula | Treat- | Final |
| Age | Gender | Site | ment | outcome |

| | | | | | |
|---|---|---|---|---|---|
| 6 | months | Male | 3 o'clock^{a} | 1 | Healed |
| 4 | months | Male | 3 o'clock | 1 | Healed |
| 14 | months | Male | 9 o'clock^{b} | 1 | Healed |
| 14 | months | Male | 3 o'clock | 1 | Healed |
| 8 | months | Male | 3 o'clock | 1 | Healed |
| 2 | months | Male | 3 o'clock | 1 | Healed |
| 144 | months | Female | 9 o'clock | 1 | Healed |

Wherein, “^{a}” refers to the anal fistula site: the health care worker observed the fistula site when the patient lay at a lying posture. The health care worker observed the fistula site by using the anal as the center, the reproductive organ was at 12 o'clock, and the fistula site was at 3 o'clock relative to the anal (i.e. the fistula was at the right of the anal).
^{b}refers to the anal fistula site: the health care worker observed the fistula site when the patient lay at a lying posture. The health care worker observed the fistula site by using the anal as the center, the reproductive organ was at 12 o'clock, and the fistula site was at 9 o'clock relative to the anal (i.e. the fistula was at the left of the anal).

As shown in Table 2, the present example showed that the pharmaceutical composition was effective for the treatment of anal fistula in children from 2 to 144 months of age. Although the gender and the fistula site were different from each child, all children were healed after treated once by using the pharmaceutical composition.

The reasons that the pharmaceutical composition of this invention is used for treating the fistula in children instead of adults are described as following. Published studies have reported the efficacy of fibrin glue treatment in adults with fistula. The success rates of the fibrin glue treatment in adults with fistula ranging from 61 to 83.3%, with follow-up ranging from 6 to 22 months. Compared to this invention, the success rate of the fibrin glue therapy in adults is lower which may cause by the following reasons: 1) the complex structure of the fistula in adults; 2) the tracts can be longer and the path can be curved in adults with fistula; 3) the fistula in adults may has many openings. Therefore, the fibrin glue cannot fully filled and sealed the tracts and openings of fistula in adults, and thus the success rate is low. Moreover, there is no study use fibrin glue for treating fistula in children. Compared to the published studies, the pharmaceutical composition of this invention is not a commercial fibrin glue. The pharmaceutical composition is prepared from the commercial fibrin glue and adjusting the coagulation time and the application method by the method described herein. Therefore, the volume, the times of dilution, the coagulation time and the applied method of the pharmaceutical composition can be adjusted according to the morphology, the type and the size of the fistula in children, so as to be suitable for treating the fistula in children. The published studies of fibrin glue treatment for adult anal fistula are listed as the following table 3.

TABLE 3
published studies of fibrin glue treatment for adult anal fistula

| | Fibrin | | Patient | Success | Follow-up | |
|---|---|---|---|---|---|---|
| Author | glue type | Year | number | rate (%) | (median) | |
| Cintron et al. | Commercial/ | 2000 | 79 | 61 | 12 | months |
| | Autologous^{a} | | | | | |
| Park et al. | Commercial^{b} | 2000 | 29 | 69 | 6 | months |
| Lindsey et al. | Commercial | 2002 | 42 | 63 | 17 | months |
| Sentovich | Commercial/ | 2003 | 48 | 68.8 | 22 | months |
| | Autologous | | | | | |
| Maralcan et al. | Commercial | 2006 | 36 | 83.3 | 54 | weeks |

Wherein, “^{a}” means that there were two types of fibrin glue used in that study. One was commercialized, and the other one was the fibrinogen and the thrombin separated from the blood of the patient. “^{b}” means that the fibrin glue was commercialized.

As shown in Table 3, no matter the fibrin glue was commercialized or separated from the patient, when the fibrin glue was used for the fistula treatment in adults, the success rate was between 61-83%, and the follow-up is between 6-22 months. However, the pharmaceutical composition of this invention is used for the fistula treatment in children with a success rate of 100%. The reasons which cause the different efficacy of adults and children already describe above, and will be omitted here.

As mentioned above, the pharmaceutical composition, the use and the method of this invention can enhance the success rate, and decrease the difficulty in fistula treatment in children, and thus avoid the side effects including prolonged healing, anal fistula recurrence, and anal incontinence.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A pharmaceutical composition for use in fistula treatment in children, wherein the pharmaceutical composition comprises a fibrinogen, a thrombin and a coagulation factor XIII, and the dose of the fibrinogen is between 20 mg/mL and 200 mg/mL, the dose of the thrombin is between 1 IU/mL and 20 IU/mL, the dose of the coagulation factor XIII is less than or equal to 10 IU/mL, and the fistula in children is anal fistula or urethra fistula.

2. The pharmaceutical composition for use according to claim 1, wherein the pharmaceutical composition is applied to the fistula in children by filling the inside of fistula from one end until another end is filled.

3. The pharmaceutical composition for use according to claim 2, wherein the pharmaceutical composition is applied to the fistula in children by using a syringe.

4. The pharmaceutical composition for use according to claim 3, wherein the fibrinogen and the thrombin are mixed to form the pharmaceutical composition just before the pharmaceutical composition is applied to the fistula in children.

5. The pharmaceutical composition for use according to claim 4, wherein the pharmaceutical composition is applied to the fistula in children as a liquid dosage form or a gel dosage form.

6. The pharmaceutical composition for use according to claim 4, wherein a volume mixing ratio of the fibrinogen and the thrombin is 1:1.

7. The pharmaceutical composition for use according to claim 1, wherein the pharmaceutical composition further comprises a calcium chloride solution.

8. The pharmaceutical composition for use according to claim 1, wherein the pharmaceutical composition is used for children under or equal to 12 years old.

9. A pharmaceutical composition for fistula treatment in children, wherein the pharmaceutical composition comprises a fibrinogen, a thrombin and a coagulation factor XIII, and the dose of the fibrinogen is between 20 mg/mL and 200 mg/mL, the dose of the thrombin is between 1 IU/mL and 20 IU/mL, the dose of the coagulation factor XIII is less than or equal to 10 IU/mL, and the fistula in children is anal fistula or urethra fistula.

10. The pharmaceutical composition according to claim 9, further comprises a pharmaceutically acceptable carrier, the pharmaceutically acceptable carrier is water, phosphate buffer, glycerol, sucrose ester, gelatin or polysorbate.

11. The pharmaceutical composition according to claim 10, further comprises a calcium chloride solution.

12. The pharmaceutical composition according to claim 9, wherein the fibrinogen is a human fibrinogen purified from a nature source, a transgene-human fibrinogen or a recombinant human fibrinogen.

13. The pharmaceutical composition according to claim 9, wherein the thrombin is a human thrombin purified from a nature source, a transgene-human thrombin or a recombinant human thrombin.

14. A method for fistula treatment in children, the method comprises a step of applying a pharmaceutical composition to the fistula in children, wherein the pharmaceutical composition comprises a fibrinogen, a thrombin and a coagulation factor XIII, and the dose of the fibrinogen is between 20 mg/mL and 200 mg/mL, the dose of the thrombin is between 1 IU/mL and 20 IU/mL, the dose of the coagulation factor XIII is less than or equal to 10 IU/mL, and the fistula in children is anal fistula or urethra fistula.

15. The method according to claim 14, wherein the pharmaceutical composition is applied to the fistula in children by filling the inside of fistula from one end until another end is filled.

16. The method according to claim 15, wherein the pharmaceutical composition is applied to the fistula in children by using a syringe.

17. The method according to claim 16, wherein the fibrinogen and the thrombin are mixed to form the pharmaceutical composition just before the pharmaceutical composition is applied to the fistula in children.

18. The method according to claim 17, wherein the pharmaceutical composition is applied to the fistula in children as a liquid dosage form or a gel dosage form.

19. The method according to claim 17, wherein a volume mixing ratio of the fibrinogen and the thrombin is 1:1.

20. The method according to claim 14, wherein the pharmaceutical composition is used for children under or equal to 12 years old.
